(19) United States
(12) Patent Application Publication
(10) Pub. No.: US 2023/0242965 A1
(43) Pub. Date: Aug. 3, 2023

(54) METHODS AND ASSEMBLIES FOR SAMPLE ANALYSIS

(71) Applicant: Charm Sciences, Inc., Lawrence, MA (US)

(72) Inventors: Robert J Markovsky, Brentwood, NH (US); David Legg, Amesbury, MA (US)

(73) Assignee: Charm Sciences, Inc., Lawrence, MA (US)

(21) Appl. No.: 17/927,472

(22) PCT Filed: May 28, 2021
(86) PCT No.: PCT/US21/34791

§ 371 Date: Nov. 23, 2022

Related U.S. Application Data
(60) Provisional application No. 63/031,158, filed on May 28, 2020.
(60) Provisional application No. 63/031,628, filed on May 29, 2020.

(51) Int. Cl. C12Q 1/34; C12N 9/14

(52) U.S. Cl. CPC C12Q 1/34; C12N 9/14; C12Y 306/01005

(57) ABSTRACT

A method of preparing for analysis of a sample is shown and described. In one embodiment, the method includes soaking at least one sample collector in an apyrase solution, and removing the apyrase solution. The result is preparing the sample collector by removing adenosine triphosphate from the sample collector.

This application claims the benefit of PCT US2021/34791, filed May 28, 2021; U.S. provisional application No. 63/031,158, filed May 28, 2020; and U.S. provisional application No. 63/031,628, filed May 29, 2020, all of which are incorporated herein by reference in their entireties.

### FIELD OF THE TECHNOLOGY

The present disclosure relates generally to the preparation of sample analysis, and more particularly to improved sample collector devices, methods, and assemblies.

### BACKGROUND

Various types of testing apparatuses may detect the presence of one or more analytes in a sample. A test swab, or similar swab device, is one type of apparatus useful and cost-effective for the detection of analytes. Traditional swab devices include an application end, for instance a foam tip, rolled cotton, or the like, secured on a shaft opposing an operation handle. Conventional methods of assembling swab devices for sensitive testing require cumbersome personnel demands, and further present contamination and/or failure concerns.

Therefore, Applicant desires systems and methods for preparing swab devices, and the like, for downstream testing usage to improve performance and minimize contamination, without the drawbacks presented by traditional systems and methods.

### Summary and Description of Embodiments

In the following description, like reference characters designate like or corresponding parts throughout the several views. Also in the following description, it is to be understood that such terms as “forward,” “rearward,” “left,” “right,” “upwardly,” “downwardly,” and the like are words of convenience and are not to be construed as 343597.doc 1 limiting terms. Further, the following summary is intended to summarize certain embodiments of the present disclosure. Embodiments will be set forth in more detail in the figures and description of embodiments below. It will be apparent, however, that the description of embodiments is not intended to limit the present inventions, the scope of which should be properly determined by the appended claims.

In accordance with the present disclosure, devices and systems are provided for the preparation and/or packaging of sample collection devices, including but not limited to swab devices. This disclosure provides improved swab devices and procedures that are convenient, efficient, and safe for the user, particularly when preparing numerous individual test swabs for sample analysis.

In one embodiment, a method of preparing for analysis of a sample includes exposing at least one sample collector to an apyrase solution, thereby defining an apyrase-absorbed sample collector; and withdrawing the apyrase solution about the apyrase-absorbed sample collector.

In certain examples the method may include substantially destroying the apyrase solution about the sample collector. The method may include substantially providing a sample collector substantially free of a residual apyrase enzyme. The method may include exposing the sample collector to a swab soak solution. The method may include withdrawing the swab soak solution about the swab-soaked sample collector.

In particular examples, a sample collector is provided from exposing at least one sample collector to an apyrase solution, thereby defining an apyrase-absorbed sample collector; and withdrawing the apyrase solution about the apyrase-absorbed sample collector.

In one embodiment, a method of preparing for analysis of a sample includes soaking at least one sample collector in an apyrase solution, thereby defining an apyrase-absorbed sample collector; mechanically substantially removing the apyrase solution about the apyrase-absorbed sample collector; submerging the sample collector in an swab soak solution, thereby defining a swab-soaked sample collector; and mechanically substantially removing the swab soak solution about the swab-soaked sample collector.

In one example, preparing the sample collector for analysis of the sample may include removing adenosine triphosphate from the sample collector. The method may include mechanically substantially removing the apyrase solution includes spinning the apyrase-absorbed sample collector. The method may include substantially destroying the apyrase solution about the sample collector. The method may include substantially destroying the apyrase solution includes providing a sample collector substantially free of a residual apyrase enzyme. The method may include substantially removing the apyrase solution includes adjusting a pH for inactivating the apyrase enzyme. The method may include substantially removing the apyrase solution includes heating the sample collector for a predetermined temperature and time profile. The method may include autoclaving, or the like, the sample collector. The method may include spinning the swab-soaked sample collector, for instance centrifuging, or the like, the swab-soaked sample collector. The method may include soaking a plurality of sample collectors having a sample collector tip in the apyrase solution, and submerging a plurality of sample collectors having a sample collector tip in a swab soak solution.

In particular examples, method may include extracting a liquid from an organic matter containing apyrase and mixing the liquid with a first solution; removing a first supernatant; mixing a second solution into the first supernatant and extracting a precipitate containing apyrase; and performing dialysis on the precipitate containing apyrase.

In particular examples, the method may include mixing a third solution with the precipitate containing apyrase thereby creating an apyrase solution. The first solution may include an organosulfur thiourea and the second solution comprises ammonium sulfate. The method may include introducing a buffer fourth solution thereby releasing a pellet formation from a surface.

In particular examples, a sample collector is provided from soaking at least one sample collector in an apyrase solution, thereby defining an apyrase-absorbed sample collector; mechanically substantially removing the apyrase solution about the apyrase-absorbed sample collector; submerging the sample collector in an swab soak solution, 343597.doc 3 thereby defining a swab-soaked sample collector; and mechanically substantially removing the swab soak solution about the swab-soaked sample collector.

In one embodiment, a method includes soaking at least one sample collector in an apyrase solution; mechanically substantially, including fully, partially, and a combination thereof, removing the apyrase solution; submerging the sample collector in an swab soak solution; and mechanically substantially, including fully, partially, and a combination thereof, removing the swab soak solution.

In particular examples, preparing the sample collector for analysis of the sample comprises removing adenosine triphosphate from the sample collector. The method may include soaking a plurality of sample collectors having a sample collector tip in the apyrase solution. The method may include submerging a plurality of sample collectors having a sample collector tip in an allergiene swab soak solution. The method may include mechanically substantially, including fully, partially, and a combination thereof, removing the apyrase solution by spinning the apyrase-absorbed sample collector. The method may include mechanically substantially, including fully, partially, and a combination thereof, removing the swab soak by spinning the swab-soaked sample collector.

In one example, the method includes extracting a liquid from an organic matter containing apyrase and mixing the liquid with a first solution; removing a first supernatant; mixing a second solution into the first supernatant and extracting a precipitate containing apyrase; and performing dialysis on the precipitate containing apyrase.

In one example, the method includes mixing a third solution with the precipitate containing apyrase thereby creating an apyrase solution. The method may include conducting at least one sterility check. The method may include monitoring an aerobic count peel plate. The method may include monitoring a yeast-mold peel plate.

The method may include soaking a swab in the apyrase solution. The method may include extracting a liquid from a russet potato. The method may include isolating apyrase enzyme from a russet potato, or any vegetable, or similar organic matter.

In particular examples, the method may include maintaining the organic material at temperature at about one to about ten degrees Celsius prior to extracting the liquid. The method may include shielding the organic material from a light exposure prior to extracting the liquid. The first solution may include an organosulfur thiourea, for instance the organosulfur thiourea being a 1-phenyl-2-thiourea.

In some examples, the second solution, for instance a detergent rinse, comprises ammonium sulfate. The concentration of the ammonium sulfate comprises about 90% concentration. The third solution may comprise sodium acetate. The third solution may comprise a pH of about 6.5. The method may include adjusting the pH level with HCl. The method may include introducing a fourth solution thereby releasing a pellet formation from a surface. The method may include introducing an ammonium sulfate fourth solution. The concentration of the ammonium sulfate may comprise about 45% concentration.

In another embodiment, a method for preparing for analysis of a sample comprises soaking at least one sample collector in an apyrase solution, thereby defining an apyrase-coated sample collector; spinning the apyrase-coated sample collector submerging the sample collector in an swab soak solution, thereby defining a swab-soaked sample collector; and spinning the swab-soaked sample collector.

In particular examples, preparing the sample collector for analysis of the sample may comprise removing adenosine triphosphate from the sample collector. The method may include soaking a plurality of sample collectors having a sample collector tip in the apyrase solution. The method may include submerging a plurality of sample collectors having a sample collector tip in an allergiene swab soak solution.

As shown and described herein, the sampling probe may include a swab device having a handling portion and a sampling distal end. Those skilled in the art having the benefit of this disclosure will recognize a variety of swab test sample apparatus elements and embodiments useful and compatible with any of the methods and systems shown and described herein. Further, various types of testing apparatuses helpful to detect the presence of analytes may be used for any of the examples and embodiments herein. For instance, those of ordinary skill in the art having the benefit of this disclosure will recognize that a variety of testing instruments, devices, and systems may be in communication with any of the inventions herein, including but not limited to, TEST DEVICE, METHOD AND ASSEMBLY (U.S. application Ser. No. 15/434,399); SAMPLING METHOD AND DEVICE (U.S. Pat. No. 7,993,871); METHOD FOR ADJUSTING ANTIBIOTIC SENSTIVITY TO A TEST CULTURE (U.S. Pat. No. 7,897,365); INHIBITION ASSAY METHOD AND DEVICE FOR DETECTION OF ANTIBIOTICS (U.S. Pat. No. 8,476,064); LATERAL FLOW ASSAY ANALYSIS (U.S. application Ser. No. 13/819,064); IMPROVED LUMINOMETER AND CHAMBER (U.S. application Ser. No. 14/154,516); DETECTION SENSOR SYSTEMS AND METHODS (U.S. application Ser. No. 14/207,896); DYNAMIC PLATE READER (U.S. application Ser. No. 14/480,994); LUMINOMETER AND CHAMBER (U.S. application Ser. No. 14/662,825); METHOD AND APPARATUS FOR REDUCING LUMINESCENT TEST RESULT INTERFERENCES (U.S. Pat. No. 8,663,975); RESEALABLE MOISTURE TIGHT CONTAINER (U.S. Pat. No. 9,493,288); Photometer for use with a test sample holder (US Design Patent No. D393,601), U.S. Pat. Nos. 5,965,453; 5,827,675; 5,985,675; 6,180,395; 6,319,446; 6,475,805; 7,097,983; 7,410,808; 7,785,899; 7,785,899; 7,897,365; 8,481,334; 8,592,171; 8,592,171; 8,592,171; and 9,057,724, any of the useful testing instrument features and elements are incorporated herein by reference.

In particular examples, the swab test sample apparatus may be a sensitive field swab device to receive sample from a source, including from a surface, from a human or animal, or from any sample collection and may be housed in a self-supported housing.

Those skilled in the art having the benefit of this disclosure will recognize additional swab test sample apparatus embodiments useful and compatible with any of the methods and systems shown and described herein.

In particular embodiments, this disclosure provides systems and methods to synthesize an apyrase enzyme for the use of detecting organic matter.

In certain examples, the organic material that contains apyrase may be stored between zero and ten, including at about one to about seven degrees Celsius, although those skilled in the art having the benefit of this disclosure will recognize a variety of storage temperatures and environments. Further, the organic material may be shielded from light until ready to use. In certain examples, liquid is removed from organic material, and the resulting liquid mixed into a first solution.

In particular examples, if the organic material is a vegetable or the like, juicing may satisfy the liquid removal process. In particular examples wherein the organic material is a russet potato, then the potatoes may be promptly juiced after slicing, for instance to not allow the potatoes to brown. In some embodiments, the first solution contains an organosulfur thiourea, such as 1-phenyl-2-thiourea, or the like. In other embodiments, the second solution comprises acetone and an organosulfur thiourea.

In particular examples, the solution may settle after the organic matter has been processed and mixed. In some examples, the contents are settled from thirty minutes to three hours, however those skilled in the art having the benefit of this disclosure will recognize a variety of settling time frames.

In particular examples, a supernatant is then removed from the solution. The supernatant may be mechanically manipulated, for instance spun in a centrifuge. In some embodiments, removing the supernatant includes processing in a centrifuge, for instance at a temperature of about one to about seven degrees Celsius and at about 7,000 RPM for about thirty minutes, or the like. Once separated, the supernatants may be pooled and pellets discarded.

In some embodiments, further separation may be performed by using a second solution to mix with the supernatant. In some embodiments, an ammonium sulfate solution may be added and mixed into the supernatant for about thirty minutes to about three hours at a temperature between about one to about seven degrees Celsius. The supernatant may then be mechanically manipulated, for instance spun in a centrifuge. In some embodiments, the centrifuge may spin at about 10,000 RPM for about thirty minutes. Upon separation, the supernatant may be removed and the pellets discarded.

In some embodiments, the resulting supernatant from the separation(s) may be combined with a third solution. In some embodiments, this third solution contains an inorganic salt. The inorganic salt may be ammonium sulfate, or the like. The solution, if containing ammonium sulfate, may be about a 90% (30.2 g/100 ml) concentration of ammonium sulfate. In particular examples, the third solution may be mixed with the supernatant. In some embodiments, the mixture should be stirred overnight, or similar time frame. The resulting mixture may be separated via mechanical manipulation, or the like, such as by a centrifuge spinning at about 10,000 rpm for about thirty minutes, or the like. In certain examples the apyrase is now contained in the pellets and the supernatant may be removed.

In some embodiments, a fourth buffer solution is introduced. In some embodiments, the fourth buffer solution may be a sodium acetate buffer at about 10 mM. Further, the resulting solution can be a pH of 6-7. In particular examples, the buffer may be made in the lab by mixing a solvent, usually water, with a solute. In some embodiments, the process combines water with sodium acetate trihydrate. The solute may be mixed into the solvent until the solute has completely dissolved. In one embodiment, about 1000 mL of water is mixed with about 68.05±0.10 g until completely dissolved before being mixed into about forty eight liters of water. If the resulting solution's pH is either higher or lower than 6-7 pH, then an acid or base may be added to correct. In particular examples, about 1M HCl is used to modify the pH of the solution. In some embodiments, 1M HCl is used to make the pH solution fall with a pH of 6.50±0.05.

The pellets may be combined, or at least aligned together, in the same container. If the pellets have been centrifuged, or the like, to cause the pellet to stick to a surface, including but not limited to the bottom, then a fourth solution may be introduced to release the pellet. In some embodiments, a sodium acetate buffer can perform this reconstitution. In particular examples, Applicants have discovered the resulting solution may be a milk-beige color.

In particular examples, the pellets may then be dialyzed. In some embodiments, dialysis tubing may be incorporated into the process as recognized by those skilled in the art having the benefit of this disclosure.

In particular examples, the dialysis of reconstituted enzyme pellets may be performed with the fourth solution at about one to about seven degrees Celsius. In some embodiments, the dialysis buffer may be regularly replaced. For example, the dialysis buffer may be changed after the first hour and then every subsequent two hours. To test conductivity, about a five mL sample of 10 mM sodium acetate buffer may be set aside between the changing of the dialysis buffer. In some embodiments, the dialyzing process may require up to about twenty-four hours, or the like.

In particular examples, upon completion of dialysis, the dialyzed apyrase may be combined and centrifuged at about 10,000 rpm for about thirty minutes. Once complete, the apyrase may be in an unpurified state. In particular purification examples, the apyrase may be purified by a purification process such as liquid chromatography, or any similar process. The apyrase may be frozen after the post-dialysis extraction until it is ready for subsequent use. In some embodiments, the apyrase is frozen at about negative twenty degrees Celsius.

In particular methods of use, examples include calculating a predetermined amount of apyrase. Using the graduated cylinder, the predetermined amount of AG-SS may be measured into a bottle having a cap. Next, a volume of the AG-SS in the same amount as the Apyrase meant to be added so that when mixed, the resulting volume is equivalent to the volume of AG-SS originally used. This resulting solution may be referred to as the apyrase solution. In some embodiments, the bottle may be inverted at least one time, including a plurality of inversions. Then, a pump may divide the solution into about 0.2 um CN membrane filter units. If desired, a sterility check may be performed. In some embodiments, the sterility check may include monitoring any of the aerobic count peel plates, yeast-mold peel plates, a combination thereof, or the like as shown and described herein.

In some embodiments, to prepare apyrase for adding to organic matter testing, an apyrase solution may soak any of the sample collectors, including swabs and the like, as shown and described herein. In some embodiments the swabs contain cotton, foam, or both. In some embodiments for soaking the swabs, all materials used can be autoclaved.

In certain embodiments, the sample collectors shown and described herein may be exposed to a soak solution to define a swab-soaked sample collector. For instance, one, or a plurality, of sample collector(s) may be submerged or sprayed, either fully or partially, in a swab soak solution. Those skilled in the art having the benefit of this disclosure may recognize a variety of soak solutions. In one example, the soak solution is an ALLERGIENE (ALLERGIENE is a registered trademark of Charm Sciences, Inc. of Lawrence, Mass.) swabbing solution, which rapidly and efficiently treats any of the sample collectors shown and described herein. For instance, Applicant has unexpectedly discovered advantages of the ALLERGIENE swabbing buffer solution removing residual, trace, or the like, of unwanted and/or remaining elements generated by any of the processes and methods herein. The ALLERGIENE swabbing buffer solution may include any composition, process, or the like described in U.S. Pat. No. 8,663,975 (Method and Apparatus for Reducing Luminescent Test Result Interference); U.S. Pat. No. 8,236,515 (Method for Detecting ATP); U.S. Pat. No. 7,824,878 (Sensitive Method for Detecting Low Levels of ATP); U.S. Pat. No. 7,494,781 (Sensitive Method for Detecting Low Levels of ATP); U.S. Pat. No. 7,132,249 (Method of determining Allergenic Food on Surfaces); U.S. Pat. No. 6,055,050 and further described in U.S. Pat. No. 5,965,453 (Test Apparatus, System and Method for the Detection of Test Samples); U.S. Pat. No. 5,985,675 (Test Device for Detection of an Analyte) and U.S. Reissue patent application Ser. No. 10/014,154; U.S. Pat. No. 6,180,395 (Reagent Chamber for Test Apparatus and Test Apparatus), all of which are incorporated herein by reference in their entireties.

Further, in certain embodiments, the sample preparations are utilized for testing, and the like, with a luminescence reader, or the similar device. The luminescence reader may include any luminescence reading device that detects relative light unites (RLU's) such as by using a photodiode, or the like, as with a photomultiplier based luminometer. The luminescence reader may, for example, be in the format of the NOVALUM II-X System, NOVALUM, NOVALUM2, LUMINATOR-K, LUMINATOR-T, and FIREFLY readers (NOVALUM, LUMINATOR, and FIREFLY are trademarks of Charm Sciences, Inc. of Lawrence, Mass.). In these embodiments, the test apparatus provides a user with the luminescence emission count, in RLU's, of a test sample. The luminescence reader and/or vial sampling element may include any device, configuration, or the like described in U.S. Pat. No. 9,568,413 (Luminometer and Chamber) and U.S. Pat. No. 8,663,975 (Method and Apparatus for Reducing Luminescent Test Result Interference), which are incorporated herein by reference in their entireties.

The following Examples and test data demonstrate the efficacy and unexpected advantages of the present disclosure:

### EXAMPLE

First, a 10 mM sodium acetate solution was prepared. 48 liters of reverse osmosis de-ionized water was placed in a carboy. 68.05±0.01 grams of sodium acetate trihydrate was added to a flask containing 1000 mL of reverse osmosis de-ionized water and stirred until completely dissolved. The sodium acetate solution was transferred to the carboy and mixed for 5 minutes. 1M HCl was introduced to adjust the pH of the solution to 6.50±0.05. Then, the total volume of the solution was raised to 50 liters and continue to mix for 15 minutes.

Second, 30 pounds of Russet Potatoes was stored in 4 degrees Celsius and protected from light.

Third, 60 mL of 0.7M 1-phenyl-2-thiourea (PTU) was added to the acetone in the flask, and mixed on a stir plate for 15 minutes.

Fourth, the PTU solution was transferred to a separate carboy from the sodium acetone carboy.

Fifth, the potatoes were sliced and juice collected into a beaker.

Sixth, the potato juice was introduced to the PTU flask and mixed, then allowed to settle. The sediment that formed from mixing was waste and was disposed of after juicing.

Seventh, the supernatant was introduced into a graduated cylinder before placing supernatant into bottles for centrifuge application. The supernatant was spun in 4 degrees Celsius and at 7000 rotations per minute (RPM) for 30 minutes. After cycle, supernatants were poured into a bucket and mixed while discarding the pellets.

Eighth, ammonium sulfate concentration was introduced to the supernatant to produce a 45% (25.8 g/100 mL) solution. The solution was mixed at 4 degrees Celsius for 1.5 hours before spinning the solution with a centrifuge at 10,000 RPM for 30 minutes.

The supernatant was pooled into a graduated cylinder while discarding the pellets.

Ninth, ammonium sulfate concentration to 90% (30.2 g/100 ml) was introduced with the supernatant and mixed at 4 degrees Celsius overnight. Then, the juice was introduced into the centrifuge at 4 degrees Celsius at 10,000 RPM for 30 minutes. Tenth, the supernatant was discarded (the apyrase now being in the pellet). The pellets were reconstituted with sodium acetate buffer until all pellets were reconstituted and the pellet was completely in solution. 5-10 milliliters of sodium acetate buffer was used to rinse each bottle. This rinse was introduced to the first bottle of reconstituted pellets. The bottle was inverted to mix. The solution produced a milk-beige color.

Eleventh, reconstituted enzyme pellet were dialyzed in approximately 4.0 L-8.0 L of 10 mM Sodium Acetate Buffer at 4 degrees Celsius, leaving headspace in tubing to allow enzyme to swell. The dialysis buffer was replaced after 1 hour and then after every subsequent two hours. Then dialyzed overnight at 4 degrees Celsius in the final buffer change. Upon completion, dialyzed apyrase was pooled and the enzyme centrifuged at 10,000 rpm for 30 minutes and in 4 degrees Celsius.

Twelfth, the supernatant was transferred to a storage container. The apyrase, which was in the supernatant, was frozen at −20 degrees Celsius to act as unpurified apyrase.

Thirteenth, a predetermined concentration of apyrase was calculated by the formula:

2000 ml*6.0U/ml=Total amount of units needed:12000units

12000 units/(concentration of Apyrase in U/ml)=(mlneeded of Apyrase)

Fourteenth, using the autoclaved graduated cylinder, 2000 ml of AG-SS was introduced into a screw top bottle with a cap. Then, the apyrase was introduced and a volume removed in excess of the 2000 ml mark from the bottle, so the final volume remained 2000 mL.

Fifteenth, with a vacuum pump and two 1000 ml 0.2 um CN membrane filter units, a 3 ml sample was poured into the beaker from each bottle.

Sixteenth, two aerobic count peel plates and two yeast-mold peel plates containing sample were monitored for colony development.

Seventeenth, for the soak process, apyrase solution was poured into a beaker and sample collectors were submerged. The AG swab soak solution was poured into a beaker deeper than the apyrase solution.

Eighteenth, sample collectors were introduced into the apyrase beaker soaked for 10 minutes. Then the sample collectors were mechanically spun in Honey Spinner cups for 10 minutes before removing sample collectors and place in the beaker with the Swab Soak Solution for 10 minutes. Then the sample collectors were mechanically spun in Honey Spinner cups for 10 minutes.

### Example

The following example and related table show efficacy levels for treated swab collectors shown and described herein. The testing involved a 300 uL spiked positive assay, shaken for five seconds and allowed to rest for one minute (to breakdown ATP, if present), with an AG-B positive spike at 1,000,000 RLU with ATP tablets for a 300 uL/assay.

For testing: in the control there was no apyrase treatment; test A included a 6 units/mL apyrase concentration and analyzed fresh for initial data.

TABLE 1
Initial baseline

| | Control | A |
|---|---|---|
| Before | 1052801 | 57086 |
| Before | 1013271 | 34056 |
| Before | 1027641 | 52715 |
| After | 965279 | |
| After | 1516114 | |
| After | 923504 | |
| Average: | 1083102 | 47952 |
| % change: | | −95.6% |

As illustrated in Table 1 above, the initial baseline data shows the control did not degrade during the analysis and the results of testing in Column A reflect the unforeseen advantage of absence of ATP.

Applicants' unexpectedly discovered the unanticipated advantages under the processes and methodology herein for providing sample collectors being substantially free of adenosine triphosphate, for example for subsequent analysis of a sample. For instance, alternative treatments, such as under cleanroom conditions, irradiation treatment, and gas treatment, failed to provide the efficiency and benefits of sample collectors being substantially free of adenosine triphosphate provided herein. Instead, the teachings herein remove residual adenosine triphosphate, and then in certain examples coupled with an autoclave process, produce a significantly less destructive time and temperature profile, for instance in which raw materials, including but not limited to swab tips and shafts, were not discolored or otherwise degraded. As a result, Applicants' unexpectedly discovered sample collectors being substantially free of adenosine triphosphate, a residual apyrase enzyme, or the like, thereby not negatively impacting any of the additional analysis and testing shown and described herein.

Numerous characteristics and advantages have been set forth in the foregoing description, together with details of structure and function. Many of the novel features are pointed out in the appended claims. The disclosure, however, is illustrative only, and changes may be made in detail, especially in matters of shape, size, and arrangement of parts, within the principle of the disclosure, to the full extent indicated by the broad general meaning of the terms in which the general claims are expressed. It is further noted that, as used in this application, the singular forms “a,” “an,” and “the” include plural referents unless expressly and unequivocally limited to one referent.

What is claimed is:

1. A method of preparing for analysis of a sample, said method comprising:
a. soaking at least one sample collector in an apyrase solution, thereby defining an apyrase-absorbed sample collector;
b. mechanically substantially removing said apyrase solution about said apyrase-absorbed sample collector;
c. submerging said sample collector in an swab soak solution, thereby defining a swab-soaked sample collector; and
d. mechanically substantially removing said swab soak solution about said swab-soaked sample collector.

2. The method of claim 1, whereby preparing said sample collector for analysis of said sample comprises removing adenosine triphosphate from said sample collector.

3. The method of claim 1, wherein mechanically substantially removing said apyrase solution includes spinning said apyrase-absorbed sample collector.

4. The method of claim 1, including substantially destroying said apyrase solution about said sample collector.

5. The method of claim 4, wherein substantially destroying said apyrase solution includes providing a sample collector substantially free of a residual apyrase enzyme.

6. The method of claim 1, wherein substantially removing said apyrase solution includes adjusting a pH for inactivating said apyrase enzyme.

7. The method of claim 1, wherein substantially removing said apyrase solution includes heating said sample collector for a predetermined temperature and time profile.

8. The method of claim 7, wherein substantially removing said apyrase solution includes autoclaving said sample collector.

9. The method of claim 1, wherein substantially removing said swab soak includes spinning said swab-soaked sample collector.

10. The method of claim 1, including soaking a plurality of sample collectors having a sample collector tip in said apyrase solution, and submerging a plurality of sample collectors having a sample collector tip in a swab soak solution.

11. The method of claim 1, including:
a. extracting a liquid from an organic matter containing apyrase and mixing said liquid with a first solution;
b. removing a first supernatant;
c. mixing a second solution into said first supernatant and extracting a precipitate containing apyrase; and
d. performing dialysis on said precipitate containing apyrase.

12. The method of claim 11, including mixing a third solution with said precipitate containing apyrase thereby creating an apyrase solution.

13. The method of claim 11, wherein said first solution comprises an organosulfur thiourea and said second solution comprises ammonium sulfate.

14. The method of claim 11, including introducing a buffer fourth solution thereby releasing a pellet formation from a surface.

15. A sample collector provided by the method of claim 1.

16. A method of preparing for analysis of a sample, said method comprising:
a. exposing at least one sample collector to an apyrase solution, thereby defining an apyrase-absorbed sample collector; and
b. withdrawing said apyrase solution about said apyrase-absorbed sample collector.

17. The method of claim 16, including substantially destroying said apyrase solution about said sample collector.

18. The method of claim 17, wherein substantially destroying said apyrase solution includes providing a sample collector substantially free of a residual apyrase enzyme.

19. The method of claim 16, including exposing said sample collector to a swab soak solution, and withdrawing said swab soak solution about said swab-soaked sample collector.

20. A sample collector provided by the method of claim 16.
